(19) United States
(12) Patent Application Publication
(10) Pub. No.: US 2010/0299815 A1
(43) Pub. Date: Dec. 2, 2010

(54) Shampoo goggles

(76) Inventors: Martha F. Wells, Pensacola, FL (US); Michael T. Cravatt, Molino, FL (US)

Correspondence Address: Wells Martha F., 911 Holsberry Place, Pensacola, Escambia, FL, 32534, US

(21) Appl. No.: 12/454,879

(22) Filed: May 26, 2009

(51) Int. Cl. A61F 9/02

(52) U.S. Cl. 2/440

(57) ABSTRACT

The present invention provides goggles with rubber seals around the face/eyes and comprises of adjustable elastic bands which ensures a tight liquid seal. These adjustable elastic bands are placed around each ear, thus providing full/complete eye protection and complete access to a person's scalp/hair during the cleaning/treatment process. The weep holes in the bottom edge which are ¼ inch in front of the lower rubber seals provide an outlet of any droplets or condensation that may form inside the goggles.

### BACKGROUND OF THE INVENTION

1. Field of the Invention

Applicant's invention relates to the protection of a person's eyes from any liquids, shampoos, dyes, or cleaning agents during the cleaning and/or treatment of a person's scalp/hair.

2. Background Information

Presently, the cleaning and/or treatment of a person's scalp/hair require the application of shampoo, water, and/or chemicals and the rinsing of the scalp/hair after these products have been applied to the scalp/hair. This process of cleaning the scalp/hair often creates discomfort and possible harm to the person receiving such cleaning due to the possibility of cleaning products, chemicals, and water entering the eyes while the procedure is being done. Also, there are person's for medical reasons such as post surgical procedures or eye infections who cannot allow any liquids, shampoos, dyes or cleaning agents to enter into the eyes during the cleaning/treatment of the scalp/hair.

The hair preparation industry attempts to increase the comfort of the person whose scalp/hair is being treated or cleaned by a number of primitive methods. For instance, the person receiving the cleaning and/or treatment of the scalp/hair often will attempt to hold a cloth over the eyes during the cleaning process, but may not be able to continue doing so due to weakness, physical deficits, loss of cognitive ability, the person's age such as the elderly in nursing homes or such as a young child, thus causing anxiety, pain, stress, and possible harm to the person receiving the cleaning and/or treatment of the scalp/hair.

The hair preparation industry has attempted to alleviate the discomfort and possible harm suffered by its clientele with a number of devices. For instance, U.S. Pat. No. 5,423,091 by Lange, U.S. Pat. No. 4,958,385 by Rushton, and U.S. Pat. No. 5,023,954 by Lyons, disclose the use of a headband comprised of absorbent material that absorbs liquids coming from the scalp/hair of a person; however, such devices tend to hold liquids against the person's skin, increasing the irritation caused by such liquids, and do not offer any protection from liquids splashing in the person's face, however, other devices such as U.S. Pat. No. 5,857,218 by Kuhlman, which comprises of a protective visor which covers the face and eyes. However, such devices cover portions of the person's scalp/hair which make cleaning and/or treatment of the entire scalp/hair difficult. This does not offer full eye protection such as seals around each eye, thus the possibility of leaks of liquids, shampoos and chemicals may enter into the eyes.

The present invention attempts to increase the comfort, protection, and accessibility to a person whose scalp/hair is being cleaned and/or treated by providing complete and full eye protection and complete and full accessibility to a person's scalp/hair, thus diverting all the uncomfortable and possible harmful products from entering the person's eyes.

### BRIEF DESCRIPTION OF THE DRAWINGS

FIG. 1 is a front view of the seal on the goggles

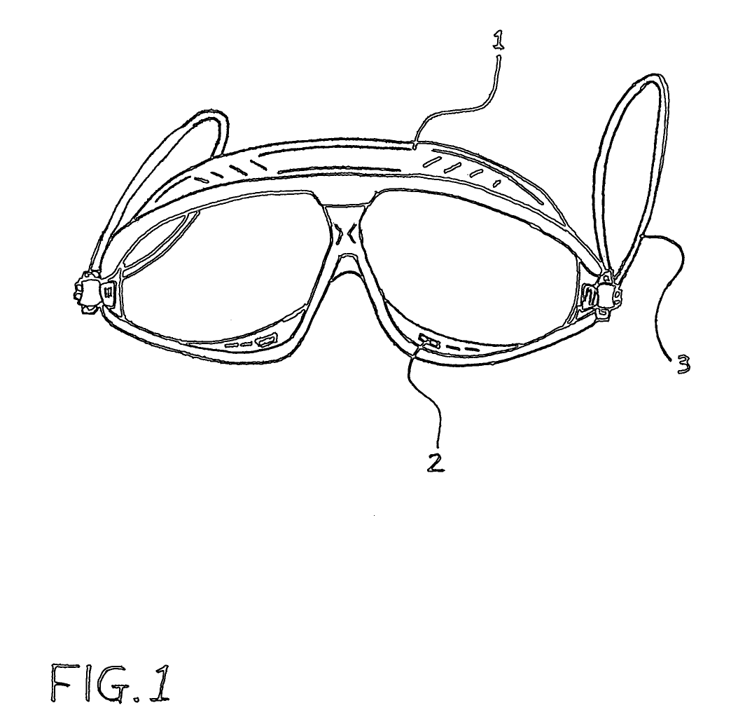

FIG. 2 is an inside view of the weep holes

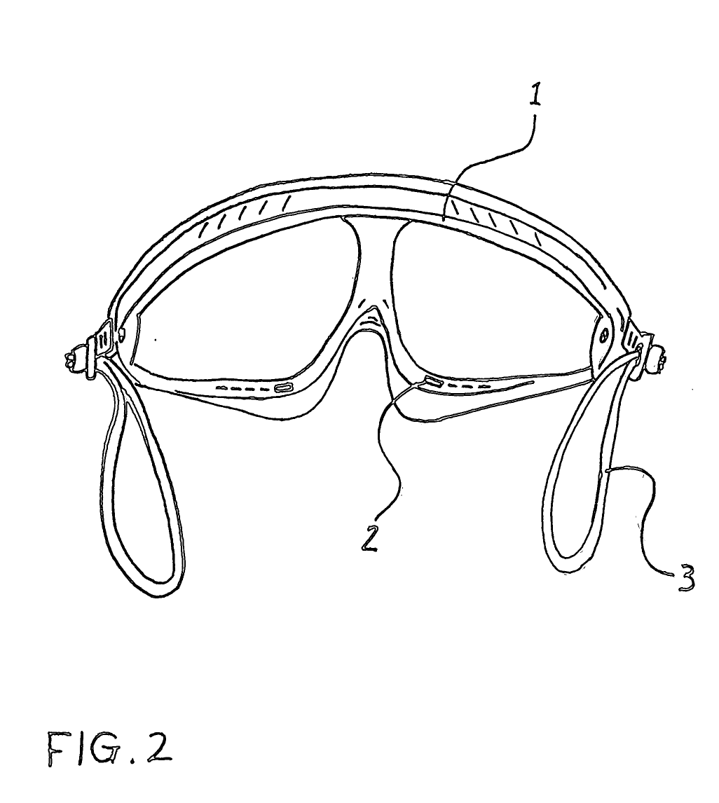

FIG. 3 is a side view of the adjustable elastic bands

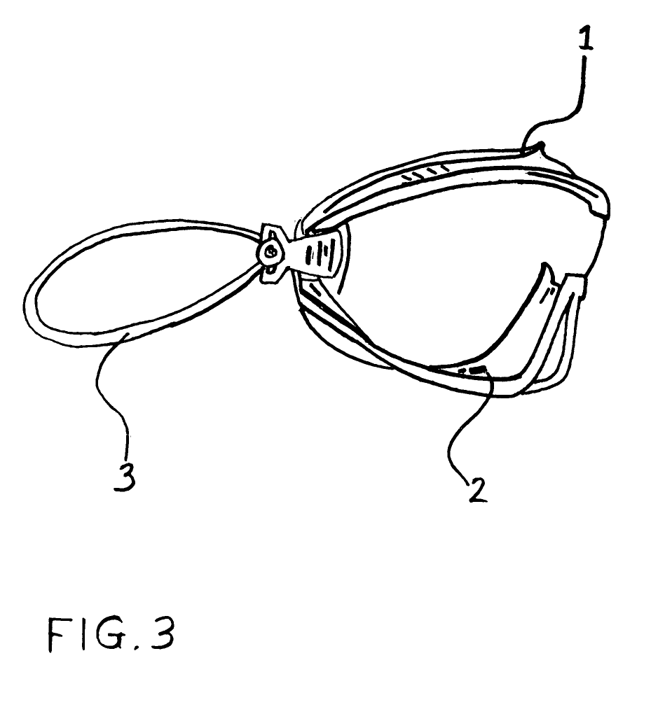

FIG. 4 is a side view of the adjustable elastic bands around the ear

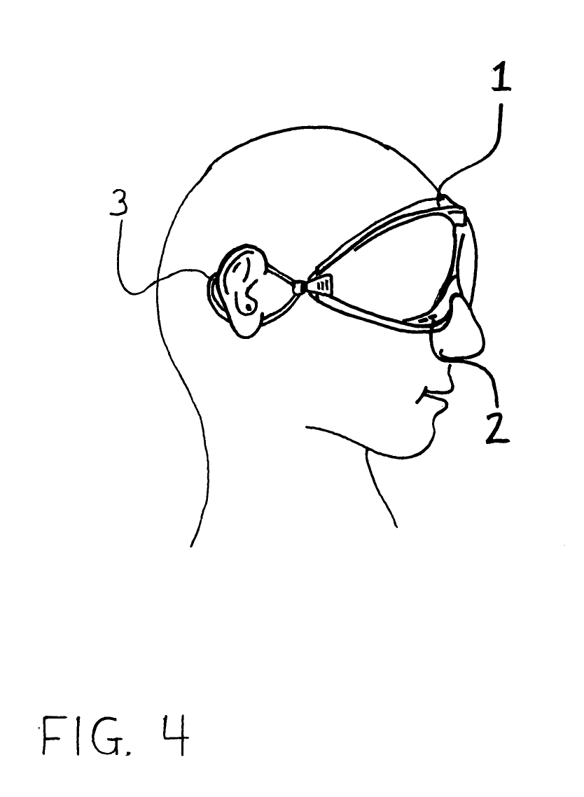

### DETAILED DESCRIPTION OF THE PREFERRED EMBODIMENT

The present invention provides complete/full eye protection and full accessibility to the scalp/hair of the person being treated. In its basic form, the invention provides (3) adjustable elastic bands that go around each ear of the person receiving scalp/hair cleaning and/or treatment. This allows full accessibility to the scalp/hair. By means of the (1) protective seal around the goggles (2) and weep holes in the bottom of the goggles which prevent any liquids, shampoos, dyes and/or chemicals from entering the eyes.

In practice, the present invention will simply be placed over the eyes, then the (3) adjustable elastic bands will be secured around each ear of the person receiving the cleaning/treatment of the scalp/hair before the cleaning/treatment begins, thus forming a complete seal around each eye for full/complete eye protection. The easy application of the adjustable elastic bands around each ear allows the person receiving the cleaning/treatment of the scalp/hair the freedom of having both hands free, eyes may remain open during the cleaning/treatment, without the possibility of any liquids, shampoos, cleaning agents, dyes or chemicals entering the eyes, thus reducing possible harm to the eyes, stress and anxiety, making the cleaning/treatment of the scalp/hair safe and comfortable.

Although, the invention has been described with reference to specific embodiments, this description is not meant to be construed in a limited sense. Various modifications of the disclosed embodiments, as well as alternative embodiments of the invention will become apparent to the persons skilled in the art upon the reference of the description of the invention. It is, therefore, contemplated that the appended claims will cover such modifications that fall within the scope of the invention.

1. A protective pair of goggles for hair treatment comprising
(A) A pair of protective goggles comprised of soft rubber seals and liquid resistant plastic lens. Adjustable elastic bands on each side of the said goggles, which fit over the person's ears that is receiving the hair treatment. Also said goggles have small weep holes in front of said rubber seal under the eyes, in case any liquid enters the goggles via the top rubber seal at the forehead will run down and out the said weep holes in the bottom edge of the goggles in front of the lower seal, thus not getting into the eyes of the person receiving the scalp/hair treatment.
(B) A process for protecting a person's eyes during scalp/hair treatment comprising steps of placing the protective goggles over the person's eyes and placing the adjustable elastic bands around the ears, thus holding the soft rubber seal on the inner side of said goggles to the persons face making a liquid tight seal to protect the eyes during the scalp/hair treatment.
